(19) United States
(12) Patent Application Publication
(10) Pub. No.: US 2023/0076866 A1
(43) Pub. Date: Mar. 9, 2023

(54) HUMAN T-LYMPHOBLASTIC LEUKEMIA/LYMPHOMA CELL STRAIN AND USE THEREOF

(71) Applicant: ZHEJIANG UNIVERSITY, Hangzhou (CN)

(72) Inventors: Jie JIN, Hangzhou (CN); Fenglin LI, Hangzhou (CN); Huafeng WANG, Hangzhou (CN); Honghu ZHU, Hangzhou (CN); Xin HUANG, Hangzhou (CN); Yi ZHANG, Hangzhou (CN); Chao HU, Hangzhou (CN); Shanshan SUO, Hangzhou (CN)

(21) Appl. No.: 17/983,420

(22) Filed: Nov. 9, 2022

Related U.S. Application Data
(63) Continuation of application No. PCT/CN2021/126508, filed on Oct. 26, 2021.

(30) Foreign Application Priority Data
Mar. 4, 2021 (CN) 202110240151.4

(51) Int. Cl. G01N 33/50; C12N 5/09

(52) U.S. Cl. CPC G01N 33/5047; C12N 5/0694; G01N 33/5011

(57) ABSTRACT

The present application discloses a human T-lymphoblastic leukemia/lymphoma cell strain named as ZYXY-T1, and its construction method and use thereof. It was conserved in China Center for Type Culture Collection (Wuhan, China) on Jan. 20, 2021, and the preservation number was CCTCC NO: C202143. The present application is obtained by separating mononuclear cells from peripheral blood of one ETP-ALL patient, and culturing the cells in vitro for continuous natural passage. The strain has the typical surface antigen expression characteristics of ETP-ALL, that is, it does not express CD1α, CD5 or CD8, and highly expresses a stem cell marker CD34, and has good proliferation ability in vitro and tumorigenesis ability in vivo; it can be used as a cell material to study the occurrence and development mechanism of ETP-ALL, and can also be used to screen and evaluate ETP-ALL drugs to guide clinical medication.

### TECHNICAL FIELD

The present application relates to the fields of biology and oncology, and relates to a human T-lymphoblastic leukemia/lymphoma cell strain, construction method and use thereof.

### BACKGROUND

Acute lymphoblastic leukemia (ALL) is a hematological malignant tumor with malignant clonal proliferation of immature lymphocytes. It is common in children, and the incidence rate of adults is lower than that of children. ALL is divided into B-cell ALL (B-ALL), accounting for about 85% of the patients, and T-cell ALL, accounting for about 15% of the patients. T-ALL is a high-risk type in ALL. Although the remission rate of patients can reach 90%-95% after combined chemotherapy, relapse occurs for nearly one-third of patients, and the overall five-year survival rate is about 50%. For the first-time refractory and relapsed patients, the current treatment methods are very limited, and the prognosis of patients is very poor.

T-lymphoblastic leukemia/lymphoma, also known as early T-cell precursor acute lymphoblastic leukemia (ETP-ALL), is a special subtype of T-ALL that has just been identified in recent years. It has the unique characteristics of surface antigen expression, that is, it lacks the expression of CD1α and CD8 of the lymphocytic surface antigen, does not express or weakly expresses CD5 surface antigen, and expresses a kind of stem cell or myeloid antigen. ETP-ALL accounts for about 15% of the patients with T-ALL, and its prognosis is even worse. Studies have shown that the complete remission rate and overall survival time of ETP-ALL patients are much lower than those of non-ETP-ALL patients, and the recurrence rate is higher than that of non-ETP-ALL patients. At present, there is no better and more effective treatment for ETP-ALL except conventional combined chemotherapy.

A cell strain is an important tool for tumor research, which not only retains the biological characteristics of a tumor, but also can be continuously subcultured in vitro, and can complete many experiments that cannot be carried out in vivo. The cell strain is the basis of tumor etiology, research and development of new tumor drugs or research of new combined tumor solutions. The research of ETP-ALL is also inseparable from cell strains, but at present, there is no ETP-ALL cell strain established from ETP-ALL patient specimens worldwide. This has greatly restricted the basic research and related clinical research of ETP-ALL. The establishment of ETP-ALL cell strains is an urgent problem to be solved in current ETP-ALL research. The establishment of an ETP-ALL cell strain is of great significance to study the pathogenesis, molecular characteristics, new drug screening, and chemotherapy regimen update of ETP-ALL, a specific type of T-ALL with poor prognosis.

### SUMMARY

The purpose of the present application is to provide the first immortalized cell strain of human T-lymphoblastic leukemia/lymphoma in the world, construction method and use thereof.

The purpose of the present application is realized through the following technical solution: a human T-lymphoblastic leukemia/lymphoma cell strain named ZYXY-T1, which was conserved in China Center for Type Culture Collection on Jan. 20, 2021 with the preservation number of CCTCC NO: C202143.

The present application also provides a progeny cell of the T-lymphoblastic leukemia/lymphoma cell strain as described above.

The present application further provides that the T-lymphoblastic leukemia/lymphoma as described above has the typical surface antigen expression characteristics of ETP-ALL of high expression of a stem cell marker CD34, and is a suspension cell.

The present application further provides the use of the human T-lymphoblastic leukemia/lymphoma cell strain as described above, which is selected from any one or more of the following:

a. study on molecular characteristics and therapeutic mechanism of ETP-ALL;

b, preparing a tumor cell model or preparing a tumor animal model; wherein, progeny cells are established from the cell strain or cells are established by transfection specific genes into the cell strain, such as luciferase reporter gene; an ETP-ALL animal model is established by subcutaneous tumor-bearing or tail vein injection;

c. screening and/or evaluating/preparing tumor therapeutic drugs; wherein, the method of screening and preparing tumor therapeutic drugs can be as follows: adding different tumor therapeutic drugs into a culture medium of the human T-lymphoblastic leukemia/lymphoma cell strain, observing the changes of cell morphology, and obtaining preliminarily effective candidate drugs; then, applying the candidate drugs to the above tumor animal model, and observing the survival period, tumor size, metastasis and other conditions as compared with the animals in a group without drug application, screening potential drugs for treating T-lymphoblastic leukemia/lymphoma;

d. developing tumor drug targets;

e. preparing tumor diagnosis products;

f. screening tumor biological therapy drugs; wherein the tumor biotherapy drug/reagent is a tumor vaccine;

g. developing bioengineering products related to tumor detection; wherein the tumor-related bioengineering products can be an ETP-ALL specific molecular diagnostic PCR kit or a fluorescence in situ hybridization kit.

The present application also provides a construction method of the human T-lymphoblastic leukemia/lymphoma, which includes the following steps: obtaining fresh peripheral blood of a patient with initial T-lymphoblastic leukemia/lymphoma; taking 6 ml peripheral blood drops and adding the blood drops into a 15 ml sterile centrifuge tube into which 6 ml of a lymphocyte separation solution is added in advance; centrifuging for 2000 rpm for 20 minutes; after centrifugation, putting a white cell precipitate of the mononuclear cell layer into a new 15 ml sterile centrifuge tube, and adding 5 ml of sterile 1×PBS resuspended cells, and centrifuging for 2000 rpm for 5 minutes; after discarding the supernatant, adding a sterile red blood cell lysate to lyse the cells at room temperature for 5 minutes, then centrifuging at 2000 rpm for 5 minutes; discarding the supernatant, adding 5 ml of an IMDM complete medium (IMDM 90%+fetal bovine serum 10%) to resuspend the cells, and centrifuging for 1500 rpm for 5 minutes; discarding the supernatant, adding 5 ml of the IMDM complete medium, and resuspending the cells; counting the cell with a cell counting plate, putting 1*10⁸ cells into a 25 cm culture flask, then adding the IMDM culture medium to 6 ml, and mixing the cells, and putting the culture flask into a constant temperature and humidity incubator at 37° C. to culture the cells; after 1 week, replacing the IMDM culture medium with a new IMDM culture medium until the cells began to proliferate.

The present application has the beneficial following effects: the human T-lymphoblastic leukemia/lymphoma cell strain of the present application can be passaged indefinitely, the cell shape in vitro is stable, and it accords with the clinical tumor biological characteristics; the human T-lymphoblastic leukemia/lymphoma cell strain originates from ETP-ALL patients, and its surface antigen conforms to the typical international definition of ETP-ALL, with high expression of the stem cell marker CD34; the human T-lymphoblastic leukemia/lymphoma cell strains can be used to study the mechanism of the occurrence and development of ETP-ALL and ALL; the cells can also be used to analyze the curative effect of new anti-leukemia drugs and combined solutions, to screen and evaluate leukemia drugs, and to guide clinical medication, and thus it is of great significance to reveal ETP-ALL, a high-risk ALL with poor prognosis.

### BRIEF DESCRIPTION OF DRAWINGS

The present application will be further explained with reference to the following examples and drawings.

FIG. 1 shows the results of microscopic observation and Wright-Giemsa staining of the human T-lymphoblastic leukemia/lymphoma cell strain;

FIG. 2 is the cell growth curve of the human T-lymphoblastic leukemia/lymphoma cell strain at different cell densities;

FIG. 3 shows the surface antigen expression result of the human T-lymphoblastic leukemia/lymphoma cell strain;

FIG. 4 shows the in vivo tumorigenicity of the human T-lymphoblastic leukemia/lymphoma cell strain, where a is the expression of CD45 in mouse peripheral blood, b is the expression of CD45 in mouse bone marrow, and c is the survival time of mice.

### DESCRIPTION OF EMBODIMENTS

The following examples will further illustrate the present application, but the present application is not limited thereto. The experimental methods without specific conditions in the following examples are usually in accordance with conventional conditions.

### Example 1 Preparation of the ZYXY-T1 Cell Strain

Primary cell culture: 6 ml of a peripheral blood sample (male) was taken from a patient with ETP-ALL diagnosed by the First Affiliated Hospital of Medical College of Zhejiang University, and leukemia mononuclear cells were immediately isolated; in the biosafety cabinet, 6 ml of the peripheral blood sample was dropped into a 15 ml sterile centrifuge tube into which 6 ml of a lymphocyte separation solution was added in advance, and the mixture was centrifuged at 2000 rpm for 20 minutes; after centrifugation, the mononuclear cell layer was put into a new 15 ml sterile centrifuge tube, 5 ml of sterile 1×PBS resuspended cells was added, and the mixture was centrifuged at 2000 rpm for 5 minutes; after discarding the supernatant, a sterile red blood cell lysate was added to lyse the cells at room temperature for 5 minutes, and then the mixture was centrifuged at 2000 rpm for 5 minutes; the supernatant was discarded 5 ml of an IMDM complete medium (IMDM 90%+fetal bovine serum 10%) was added to resuspend the cells, and the mixture was centrifuged at 1500 rpm for 5 minutes; the supernatant was discarded, 5 ml of the IMDM complete medium was added to resuspend the cells; a cell counting plate counted the cells, 1*10⁸ cells were added into a 25 cm culture flask, then the IMDM culture medium was added to 6 ml, the cells were mixed, and the culture flask was put into a constant temperature and humidity incubator at 37° C. to culture the cells; after 1 week, the IMDM culture medium was replaced with a new IMDM culture medium to remove the cell debris, and the culture continued; the culture medium was changed once a week.

Cell subculture: after 1-2 weeks of culture, the cells underwent apoptosis; the remaining cells without apoptosis were in a state of non-proliferation and non-death, and the culture medium was changed every week; when the cells were cultured for 2 months, they began to proliferate and grew in suspension; at this time, the cell culture medium was changed every 48-72 hours and the passage began; up to now, the cells have been passaged for more than 50 generations and is an immortalized cell strain.

According to the present application, cells grow in a suspension state, single cells grow, cells are round or oval, and the cell growth rate is stable. The cell stain was named ZYXY-T1, and was conserved in China Center for Type Culture Collection (address: Wuhan University, Wuhan, China) on Jan. 20, 2021, with the preservation number of CCTCC NO: C202143.

### Example 2 Biological Characteristics and Use of the Human T-Lymphoblastic Leukemia/Lymphoma Cell Strain

According to the present application, the cell ZYXY-T1 was cultured in an IMDM culture medium containing 10% fetal bovine serum, so that it could grow stably in vitro and be passaged stably; microscopically, the cells were suspended and grow individually, round or oval; wright-Giemsa staining showed that the cells were acute leukemia protocells with big hyperchromatic nuclei; by flow analysis, it was found that this cell strain has the typical surface antigen characteristics of ETP-ALL, and it is the first ETP-ALL cell strain established internationally; the cell strain can be used to study the pathogenesis of ETP-ALL, screen and/or evaluate/prepare tumor therapeutic drugs; developing tumor drug targets; preparing tumor diagnosis products; screening tumor biotherapy drugs/reagents; develop tumor-related bioengineering products, which are specifically as follows:

Morphological Observation

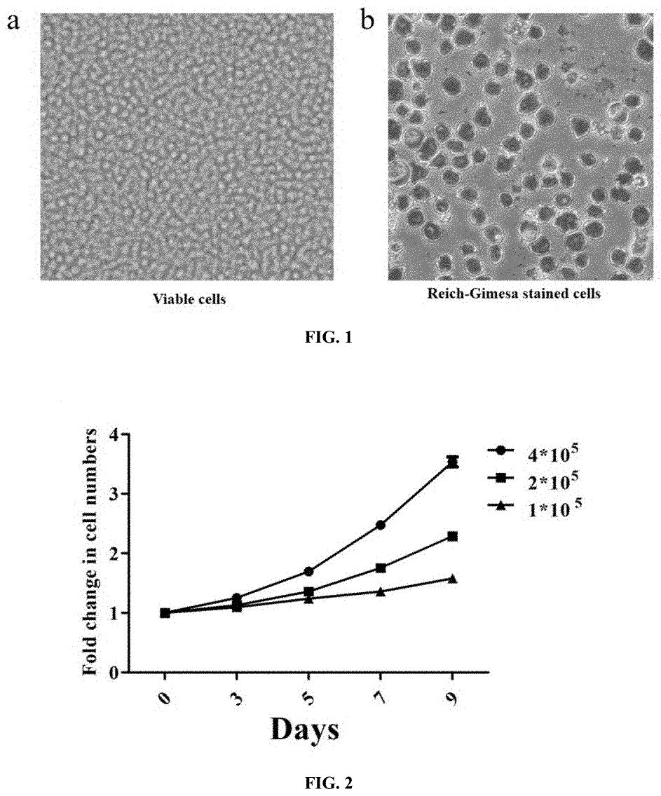

The cultured ZYXY-T1 cell strain was observed under an inverted microscope and photographed. As shown in FIG. 1a, the cells grew in a single suspension, and the cells were round or oval. 1*10⁶ cultured cells were put in a 1.5 ml EP tube and centrifuged at 1500 rpm for 5 minutes; the supernatant was discarded, 10 ul of a culture medium was added to suspend the cells, and then the slides were pushed; after the cell smear was dried, the cell smear was stained with a Gareth-Giemsa staining solution for 5 minutes, then rinsed and dried; the morphology of the cells was observed under the inverted microscope. As shown in FIG. 1b, the cells showed big hyperchromatic nuclei with a mononuclear state, and there were obvious spinous processes on the surface of the cells.

Observation of Proliferation Ability In Vitro

The cultured ZYXY-T1 cell strain was plated on a 96-well plate at the concentration of 1, 2, 4*10⁵/ml, and each well was plated with 100 P. L; at 0, 24, 48, 72 and 96 hours, 20 ul of a cell proliferation reagent MTS was added respectively; after 4 hours, the absorbance value of the 96-well plate was measured with a microplate reader, and the proliferation curves of cells at different plating concentrations were drawn by graphpad software, as shown in FIG. 2; the cells of the cell strain had good proliferation ability in vitro and showed malignant growth.

Flow Cytometry Surface Antigen Examination

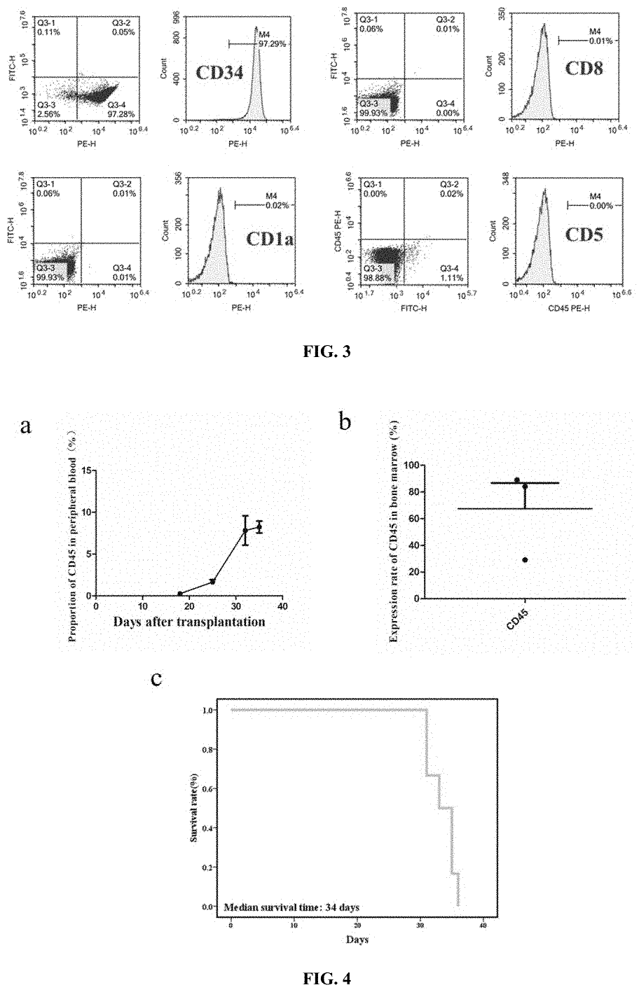

The cultured cells (1*10⁶) were divided into 5 parts, packed in 5 clean and sterile EP tubes, and centrifuged at 1500 rpm; after 5 min, the supernatant was discarded and the cells were washed with 1×PBS; the cells were centrifuged at 1500 rpm, the supernatant was discarded after 5 min, 100 ul of 1×PBS resuspended cells were added into each EP tube, the first tube without antibody, the second tube with a CD1α antibody, the second tube with a CD8 antibody, the third tube with a CD5 antibody, and the fourth tube with a CD34 antibody, each antibody added with 10 ul; the cells were incubated at room temperature for 30 min, then 1 ml of 1×PBS was added, and the tube was centrifuged at 1500 rpm for 5 min; the supernatant was discarded, and 300 ul of 1×PBS was added into each tube to resuspend the cells; the expressions of CD1a, CD8, CD5 and CD34 were measured by a flow cytometry. As shown in FIG. 3, this cell strain does not express CD1a, CD5 and CD8 antigens, but highly expresses CD34, which accords with the definition of ETP-ALL and is a typical surface antigen expression characteristic of ETP-ALL.

Observation on the Tumorigenic Ability of Cells In Vivo

The cultured ZYXY-T1 cell strain was injected into the immunocompromised NCG mice of 6-8 weeks old through the tail vein at the amount of 5*10⁶/mouse. On the 18th and 25th day after the injection of cells and when the mice were dying, 100 ul of peripheral blood of the mice was taken, and 2 ml of an erythrocyte lysate was added to lyse the peripheral blood of the mice at room temperature for 5 min, and then the mixture was centrifuged at 2000 rpm for 5 min; the supernatant was removed, and the cells were resuspended with 1 ml of 1×PBS, and were centrifuged at 2000 rpm for 5 min; after one repetition, the cells were equally divided into two parts, 300 ul of 1×PBS was added into one part, and 300 ul of 1×PBS+anti-human CD45 antibody was added into the other part; after incubation in the dark at room temperature for 30 min, 1 ml of 1×PBS was added, and then the mixture was centrifuged at 2000 rpm for 5 min; the supernatant was discarded, and 300 ul of 1×PBS was added to each tube to resuspend the cells; then, the expression of CD45 was measured by a flow cytometry; the mice were killed when they were dying, the bone marrow cells of the mice were taken and resuspended with 1×PBS, then centrifuged at 2000 rpm for 5 min, the supernatant was removed, and the cells were resuspended with 1 ml of 1×PBS and centrifuged at 200 rpm for 5 min; after one repetition, the cells were divided into two equal parts, 300 ul of 1×PBS was added into one part, and 300 ul of 1×PBS+anti-human CD45 antibody was added into the other part; after incubation in the dark at room temperature for 30 min, 1 ml of 1×PBS was added, and then the mixture was centrifuged at 2000 rpm for 5 min; the supernatant was discarded, and 300 ul of 1×PBS was added to each tube to resuspend the cells; then, the expression of CD45 was measured by a flow cytometry; finally, the survival time of mice was recorded; the results are shown in FIG. 4; after the mice were injected with cells, the expression of CD45 in peripheral blood gradually increased with time (FIG. 4a); after the mice were injected with cells, they showed a good bone marrow homing ability (FIG. 4b), and all the mice finally got sick and died, with a median survival time of 34 days (FIG. 4c). It shows that this cell strain has good tumor ability in vivo, and it is a good cell material for constructing T-lymphoblastic leukemia/lymphoma animal model. It can be used as a cell material to study the development mechanism of ETP-ALL and individualized treatment in vitro. It can also be used to screen and evaluate ETP-ALL drugs in vivo and in vitro, and guide clinical medication.

STR Identification Of cells

The cultured cell strains and the newly isolated cells of the patients were all sent to Shanghai Yihe Biology for genotyping of STR and Amelogenin loci. The results indicated that the cell strain was unique and unmatched with the existing cell strains in the world, and it was completely matched with the genotype of the STR and Amelogenin loci of the newly isolated cells from patients, that is, there was no cross-contamination during the correct culture of the cell source. The genotyping of the STR and Amelogenin loci in cells is shown in Table 1.

TABLE 1
Genotyping results of STR and Amelogenin loci of cells

| | STR information of cells for inspection | | |
|---|---|---|---|
| | Name of cells for inspection: ZYXY-T1 | | |
| Loci | Allele1 | Allele2 | Allele3 |
| D5S818 | 10 | 13 | |
| D13S317 | 9 | 9 | |
| D7S820 | 12 | 13 | |
| D16S539 | 8 | 11 | |
| VWA | 14 | 15 | |
| TH01 | 6 | 7 | |
| AMEL | X | Y | |
| TPOX | 8 | 11 | |
| CSF1PO | 12 | 12 | |
| D12S391 | 19 | 21 | |
| FGA | 23 | 24 | |
| D2S1338 | 23 | 23 | |
| D21S11 | 30 | 31 | |
| D18S51 | 14 | 19 | |
| D8S1179 | 13 | 14 | |
| D3S1358 | 14 | 17 | |
| D6S1043 | 13 | 13 | |
| PENTAE | 15 | 19 | |
| D19S433 | 13 | 13.2 | |
| PENTAD | 10 | 11 | |
| D1S1656 | 17.3 | 18.3 | |

The above examples are used to explain, but not to limit the present application. Any modifications and changes made to the present application within the scope of protection of the spirit and claims of the present application fall within the scope of protection of the present application.

What is claimed is:

1. A human T-lymphoblastic leukemia/lymphoma cell strain, wherein the cell strain is named as a human T-lymphoblastic leukemia/lymphoma cell ZYXY-T1 and had been conserved in China Center for Type Culture Collection on Jan. 20, 2021, with a preservation number of CCTCC NO: C202143.

2. The human T-lymphoblastic leukemia/lymphoma cell strain according to claim 1, wherein the cell strain is a suspension cell, which conforms to typical ETP-ALL surface antigen expression characteristics: CD1a, CD8 and CD5 are not expressed, while a stem line marker CD34 is expressed.

3. A progeny cell of the human T-lymphoblastic leukemia/lymphoma cell ZYXY-T1 according to claim 1.

4. Use of the human T-lymphoblastic leukemia/lymphoma cell strain according to claim 1, which is selected from any one or more of the following:
a. study on molecular characteristics and therapeutic mechanism of ETP-ALL;
b. preparing a tumor cell model or preparing a tumor animal model;
c. in vitro screening, in vitro evaluation or preparation of tumor therapeutic drugs;
d. developing tumor drug targets;
e. preparing tumor diagnosis products;
f. in vitro screening of tumor biological therapy drugs/reagents; and
g. in vitro development and detection of tumor-related bioengineering products.

5. The use according to claim 4, wherein an animal for preparing a tumor animal model in b is an immunodeficient mouse.
